(19) United States
(12) Patent Application Publication
(10) Pub. No.: US 2024/0268728 A1
(43) Pub. Date: Aug. 15, 2024

(54) CUVETTE

(71) Applicant: ENTIA LIMITED, London (GB)

(72) Inventors: Gerardo Jose Leon GALVEZ, London (GB); Judith BERGER, London (GB); Joseph O'CONNOR, London (GB); Toby BASEY-FISHER, London (GB); Honor JENNINGS, London (GB)

(21) Appl. No.: 18/566,860

(22) PCT Filed: May 6, 2022
(86) PCT No.: PCT/GB2022/051156

§ 371 Date: Dec. 4, 2023

(30) Foreign Application Priority Data
Jun. 4, 2021 (GB) 2108004.9

(51) Int. Cl. A61B 5/15; G01N 1/40; G01N 21/07

(52) U.S. Cl. CPC A61B 5/150343; A61B 5/150251; G01N 1/40; G01N 21/07

(57) ABSTRACT

A cuvette for collecting a blood sample, the cuvette comprising: a main body; a collection aperture formed within the main body; an ingress aperture, allowing communication between a first end of the collection aperture and an exterior of the cuvette; and a retention chamber formed within the main body and adjacent a second end of the collection chamber, which is generally opposite the first end thereof, wherein: the collection chamber has a first depth, which is the same or substantially the same across the collection chamber, from the first end to the second end thereof; and the retention chamber has a second, greater depth.

The present invention relates to a cuvette, and in particular concerns a cuvette which is adapted for collection of a fluid sample, and subsequent analysis of the fluid sample, for instance after centrifugation.

Analysis devices are known that determine an optical property of a fluid contained within a cuvette. In such devices, the cuvette is at least partially filled with a fluid sample for analysis, and placed into the device. Optical (or other) radiation is passed through the fluid, to allow properties of the fluid sample to be measured.

Examples of known types of cuvette can be seen in, for example, EP2096444A1, EP1055112A1, WO2007/008137 and GB2555403.

Optical analysis of blood and other fluid samples is utilised for a wide range of medical diagnostics, including the diagnosis of anaemia. For example, an optical analysis device can analyse a blood sample to determine haematocrit, and the presence and quantity of haemoglobin.

It is important that a cuvette can be handled easily and reliably to obtain a blood sample, particularly by users who may have reduced dexterity, coordination or vision. It is also important that, in use, the cuvette gathers a fluid sample reliably and allows analysis of the entire sample in subsequent steps.

It is an object of the present invention to provide an improved cuvette of this type.

Accordingly, one aspect of the present invention provides a cuvette for collecting a blood sample, the cuvette comprising: a main body; a collection aperture formed within the main body; an ingress aperture, allowing communication between a first end of the collection aperture and an exterior of the cuvette; and a retention chamber formed within the main body and adjacent a second end of the collection chamber, which is generally opposite the first end thereof, wherein: the collection chamber has a first depth, which is the same or substantially the same across the collection chamber, from the first end to the second end thereof; and the retention chamber has a second, greater depth.

Advantageously, in the region where the collection chamber meets the retention chamber, the collection chamber is generally planar, and the retention chamber has a depth that is greater than that of the collection chamber on both sides of the plane of the collection chamber.

Preferably, in the region where the collection chamber meets the retention chamber, the collection chamber is generally planar, and the retention chamber has a width, in a direction generally parallel with the plane of the collection chamber, which is greater than the width of the second end of the collection chamber.

Conveniently, the collection chamber has a pair of sidewalls.

Advantageously, the width of the collection chamber, defined between the sidewalls thereof, is greater at the first end of the collection chamber than at a middle portion thereof.

Preferably, the width of the collection chamber is greater at the second end thereof than at the middle portion.

Conveniently, the cuvette further comprises one or more communication passages, which are in communication with the retention chamber at a first end thereof, and are in communication with the exterior of the cuvette at a second end thereof.

Advantageously, the cuvette comprises two communication passages.

Preferably, the second ends of the two communication passages are on either side of the ingress aperture.

Conveniently, the cuvette further comprises an analysis chamber, which is in communication with the retention chamber at a first end thereof.

Advantageously, the analysis chamber is enclosed on all or substantially all sides, apart from the first end thereof.

Preferably, the analysis chamber is elongate, and is of constant or substantially constant width along its length.

Conveniently, the analysis chamber is of constant or substantially constant depth along its length.

Advantageously, the volume of the analysis chamber is the same as, or greater than, the volume of the collection chamber.

Preferably, the cuvette further comprises a junction region adjacent the retention chamber, the analysis chamber and one or more communication passages being in communication with the junction region.

Conveniently, the dimensions of the ingress aperture and collection chamber are such that, when the ingress aperture is touched to a blood sample, blood is drawn into the collection chamber through capillary action.

Advantageously, the dimensions of the collection chamber and retention chamber are such that blood drawn into the collection chamber through capillary action will stop when it reaches the retention chamber, and will not be drawn into the retention chamber.

Preferably, the main body is generally planar, and the cuvette further includes one or more protrusions which extend away from the plane of the main body, such that when the cuvette is placed on a planar surface, at least one part of the main body is raised above the surface by a distance which is sufficient to allow a person to grasp the cuvette.

Conveniently, the distance is at least 0.8 cm.

Advantageously, a pair of protrusions are provided, on opposing sides of the main body.

Preferably, the or each protrusion extends away from the plane of the main body in both directions.

Another aspect of the present invention provides a method of collecting a blood sample, comprising the steps of touching the ingress aperture of a cuvette according to any preceding claim to a blood sample, such that blood is drawn into the collection chamber of the cuvette by capillary action, to fill or substantially fill the collection chamber, without substantially entering the retention chamber.

Conveniently, the cuvette is a cuvette according to any one of the above, and the method further comprises the step of centrifuging the cuvette so that the blood in the collection chamber flows through the retention chamber and into the analysis chamber.

In order that the invention may be more readily understood, embodiments thereof will now be described, by way of example, with reference to the accompanying figures, in which:

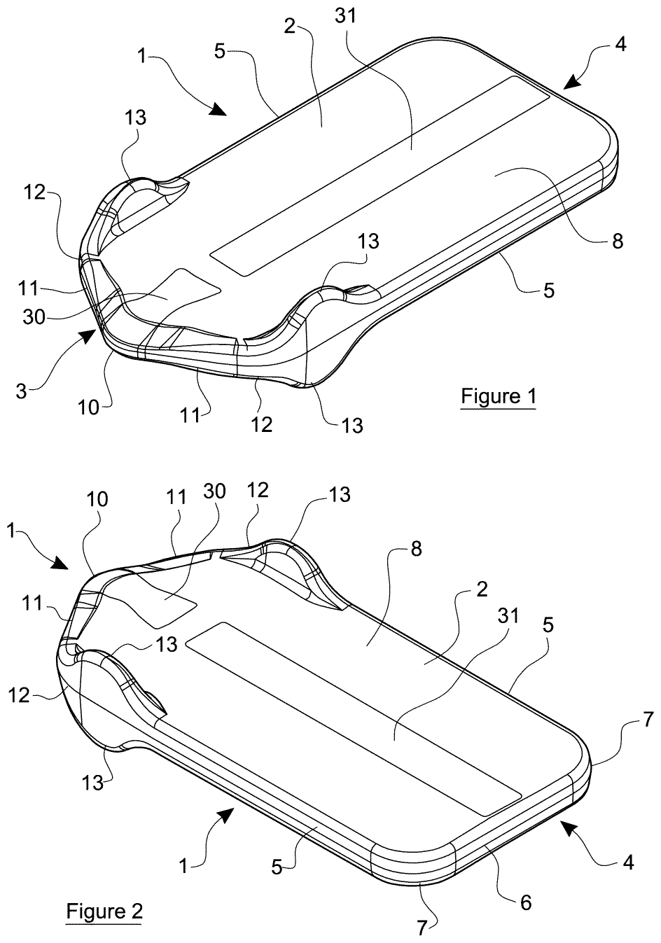

FIGS. 1 and 2 are perspective views of a cuvette according to the invention;

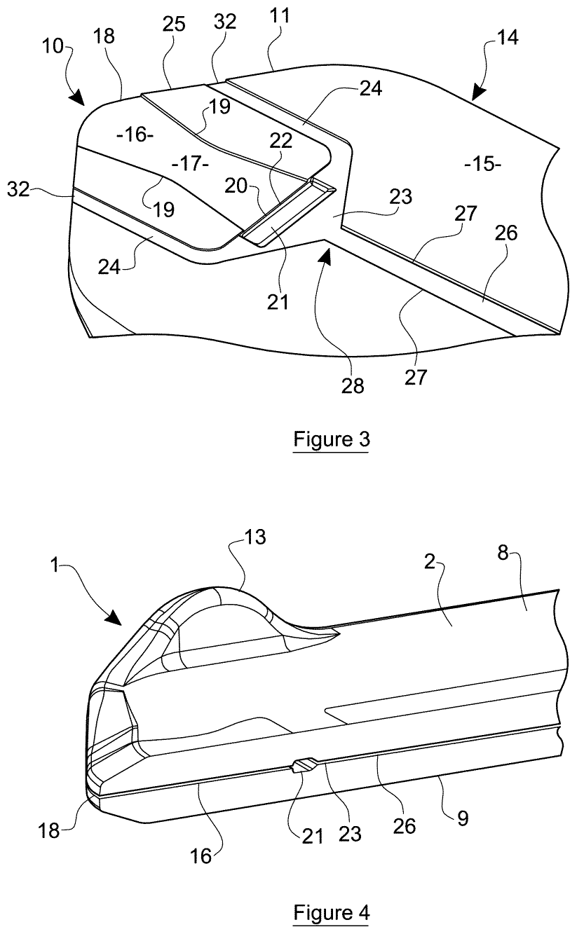

FIG. 3 is a close-up view of one half of the cuvette of FIG. 1;

FIG. 4 is a cut-away view of the cuvette of FIG. 1;

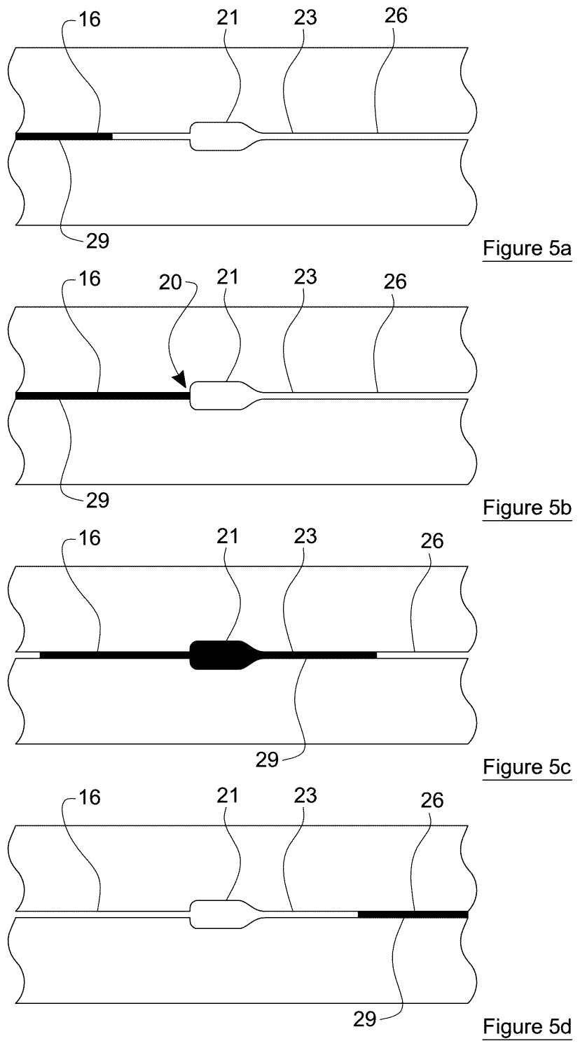

FIGS. 5a to 5d show stages in the use of the cuvette of FIG. 1; and

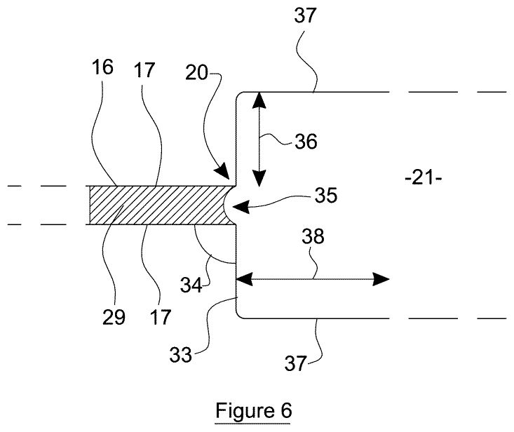

FIG. 6 is a close-up cut-away view of part of the cuvette of FIG. 1.

FIGS. 1 and 2 show front and rear perspective views, respectively, of a cuvette 1 embodying the present invention.

The cuvette 1 has a main body 2, which is generally planar. The main body 2 has a front end, generally indicated by reference numeral 3, and a rear end, generally indicated by reference numeral 4.

The main body 2 generally takes the form of a rectangle, having side surfaces 5 which are, in the illustrated embodiment, generally parallel with one another. The main body also has a rear surface 6, which is preferably generally perpendicular with the side surfaces 5. In the illustrated embodiment, there are chamfered or rounded corners 7 between the side surfaces 5 and the rear surface 6. These corners 7 increase the ease of handling of the cuvette 1.

The main body 2 also has generally planar upper and lower surfaces 8, 9, which in the illustrated embodiment are also generally parallel with one another.

At its front end 3, the cuvette 1 has a protruding apex 10, which is formed by respective angled surfaces 11. The angled surfaces 11 each protrude from a corner 12, where they meet the respective side surfaces 5, in a direction which is forwardly (i.e. in the direction generally passing from the rear end 4 to the front end 3 of the cuvette 1), until the angled surfaces 11 meet each other at the apex 10. The apex 10 preferably has a rounded or radiused shape.

At the forward end of each side surface 5, protrusions 13 project upwardly and downwardly with respect to the main body 2. In the illustrated example, the protrusions 13 project away from the plane of the main body 2, and in the example shown are generally perpendicular to the plane of the main body 2.

In the example shown the protrusions 13 are generally rounded.

If the protrusions 13 were not present, the cuvette 1 would be generally planar. If the cuvette 1 rested on a flat surface, such as a table top, the cuvette 1 would lie flat against the table top and may be relatively difficult to grasp and pick up, particularly for a user with reduced dexterity.

However, the presence of the protrusions 13 means that when the cuvette 1 is placed on a flat surface, the front end 3 of the main body 2 is lifted upward away from the surface, thus making it much more easy for a user to grasp and pick up the cuvette 1.

The skilled reader will appreciate that the protrusions need not be formed in the exact locations shown in the figures, and any suitable number of protrusions, positioned at any convenient locations on the cuvette 1, may be used in order to improve the ease with which the cuvette may be grasped and handled.

In the example shown in the figures the protrusions 13 project both upward and downward from the main body 2, so that the front end 3 of the main body 2 is lifted upward from a surface, regardless of which way up the cuvette 1 is. In other embodiments, the protrusions 13 may project in only one direction from the main body 2. In yet further examples, each protrusion projects in one direction from the main body, but the protrusions project in different directions from each other (e.g. one upwardly from the plane of the main body, and the other downwardly). This will ensure that one corner of the cuvette is always lifted upwardly from a flat surface, while minimising the weight of the cuvette.

In preferred embodiments, the presence of the one or more protrusions 13 means that, when the cuvette 1 is placed on a planar surface, at least one part of the main body 2 is raised up above the surface by a distance that will provide enough space between the main body and the surface for the cuvette 1 to be easily grasped and picked up by a user. For most users, this distance will be at least 0.8 cm, but the invention is not limited to this.

The cuvette 1 may be formed from two halves, which are manufactured separately and then attached to one another. One half may include the upper surface 8 of the cuvette 1, with the other half comprising the lower surface 9 thereof. One or both of the halves may be formed with indentations on their inner surfaces so that, when the two halves are fixed together, one or more chambers exist within the main body 2 of the cuvette 1. These chambers will be discussed in more detail below. However, it should be understood that the invention is not limited to this method of manufacture of the cuvette, and the cuvette may be formed in any other suitable or convenient manner.

Turning to FIG. 3, one half 14 of the cuvette 1 is shown in isolation. In some embodiments, indentations are formed on the inner surface of one half of the cuvette, with the inner surface of the other half being substantially smooth and/or flat. In other examples, both halves may have indentations formed on their inner surfaces, and when the cuvette is assembled then at least some of the indentations align with each other to form the chambers.

FIG. 3 shows an inner face 15 of the half 14, i.e. the face which will be within the main body 2 of the cuvette 1 when the cuvette 1 is fully assembled.

The majority of the inner face 15 is planar and parallel, presenting a flat inward-facing surface which, in the fully assembled cuvette 1, will meet and abut against the inner face 15 of the other half 14, with no or substantially no gap being present between the two inner faces 15. Where the inner face is flat in this manner, the two halves 14 will meet each other and form an effectively solid region of the main body 2.

A collection chamber 16 is formed in the region of the apex 10, at the front end 3 of the half 14. The collection chamber 16 takes the form of an indented or cut-out region, which has an inner surface 17 which is below the main regions of the inner face 15.

The inner surface 17 of the collection chamber 16 is preferably parallel with the main regions of the inner face 15, and offset by a first distance with respect thereto.

The collection chamber 16 extends to the apex 10, and has an ingress aperture 18 which preferably extends symmetrically with respect to the apex 10.

In the region of the apex 10, the thickness of the main body 2 is preferably reduced. The main body 2 may have a tapering cross-section (as shown in the figures), so that the thickness gradually reduces towards the apex 10. This will help to provide a clearly-defined “point” which can be used to collect a blood sample, and also allows a blood sample that has been collected to be seen more easily by a user. The region of reduced thickness (including any tapered portion) may, for instance, overlie around the quarter, third or half of the collection chamber 16 that is nearest to the apex 10.

The collection chamber 16 has first and second side walls 19, which extend in a generally rearward direction from the respective sides of the ingress aperture 18.

In the depicted embodiment, as the side walls 19 extend rearwardly from the ingress aperture 18, the side walls 19 taper inwardly towards one another, and then flare out again from each other. The benefit of this will be discussed in more detail below.

At its inner end 20 (i.e. the end furthest from the ingress aperture 18), the collection chamber 16 meets a retention chamber 21. The retention chamber 21 has a depth which is greater than the depth of the collection chamber 16. Where the collection chamber 16 meets the retention chamber 21, at the inner end 20 of the collection chamber 16, there is therefore a shoulder, where the depth changes relatively abruptly between the first, relatively small depth of the collection chamber 16 and the second, relatively large depth of the retention chamber 21.

In the example shown the retention chamber 21 is generally rectangular, and also extends across the entire width of the inner end 20 of the collection chamber 16. In other words, all parts of the inner end 20 of the collection chamber 16 communicate with the retention chamber 21.

In the example shown the retention chamber 21 is set into a junction region 23, which (in the depicted embodiment) surrounds all sides of the retention chamber 21, apart from the side where the retention chamber 21 meets the collection chamber 16. The depth of the junction region 23 is less than that of the retention chamber 21, and is preferably the same or substantially the same as that of the collection chamber 16.

A pair of communication passages 24 extend from the junction region 23, and communicate with the exterior of the half 14 at an outlet aperture 32. Preferably, the outlet aperture 32 of each communication passage 24 extends to a front edge 25 of the half 14. In the embodiment shown, each outlet aperture 32 is on the front edge 25, at a region which is spaced apart from the ingress aperture 18. The outlet apertures 32 may be on opposite sides of the ingress aperture 18. In the example shown, each outlet aperture 32 is on the front edge 25 partway along one of the angled surfaces 11 at the front end 3.

Preferably the two communication passages 24 are arranged symmetrically.

It is important that the communication passages 24 provide a gas flow path from the junction region 23 to an exterior of the cuvette 1, when the two halves 14 of the cuvette 1 are assembled. However, the communication passages 24 may communicate with the exterior of the cuvette 1 at any suitable location, for instance at a side surface thereof, and it is not essential that the communication passages 24 provide communication with the front end 3.

Although the depicted embodiment includes two communication passages 24, in other examples the cuvette 1 may have a single communication passage.

An analysis chamber 26 extends in a rearward direction from the junction region 23. The analysis chamber 26 preferably has a depth which is the same or generally the same as that of the collection chamber 16 and junction region 23.

The analysis chamber 26 is preferably of consistent width along its length, having opposing side walls 27 which are parallel with each other. The analysis chamber 26 is preferably of constant depth along its length. The analysis chamber 26 extends towards the rear end 4 of the half 14, and terminates at a dead end (not shown in FIG. 3) which does not allow any communication with the exterior of the cuvette 1. The analysis chamber 26 therefore preferably communicates with the junction region 23 at its top end 28, and is otherwise enclosed on all sides. The dead end of the analysis chamber 26 preferably has a squared-off shape, comprising an end surface which is at right-angles or substantially at right-angles to the length of the analysis chamber 26.

Preferably, the volume of the analysis chamber 26 is greater than the volume of the collection chamber 16. In embodiments, for example, the volume of the analysis chamber 26 may be 5%-15% greater than that of the collection chamber 16.

FIG. 4 shows a cross-sectional view of the assembled cuvette 1. As can be seen clearly in this view, the collection chamber 16 extends rearwardly from the ingress aperture 18, and has a consistent or substantially consistent depth along its length (the skilled reader will understand that, if indentations to form the collection chamber 16 are formed in both halves 14, then the total depth of the collection chamber 16 of the assembled cuvette 1 is twice the depth of the indentations formed in the two halves 14).

The collection chamber 16 communicates with the retention chamber 21, which has a greater depth. At its rearward end, the retention chamber 21 communicates with the junction region 23, which in turn communicates with the analysis chamber 26. The junction region 23 and analysis chamber 26 have the same depth, in this example, and no boundary between these two regions 23, 26 is visible in FIG. 4.

FIGS. 5a to 5d show stages in use of the cuvette 1 to collect a blood sample.

As a first step, the cuvette 1 is grasped, and the apex 10 thereof is touched to a quantity of blood. This may be obtained, for instance, through a finger prick which gives rise to a drop of blood on a user's finger.

As the ingress aperture 18 touches the blood, blood is drawn into the ingress aperture 18, and thus into the collection chamber 16, by capillary action. FIG. 5a shows a cross-sectional view of the interior of the cuvette 1, in the region of the retention chamber 21. In FIG. 5a, a quantity of blood 29 can be seen travelling along the length of the collection chamber 16. At this point, the blood 29 has not reached the retention chamber 21.

In order for blood 29 to be drawn effectively into the collection chamber 16 by capillary action, the collection chamber 16 must have a suitable depth. As the skilled reader will appreciate, this depth will depend on the dimensions (particularly the length) of the collection chamber 16, and also the material from which the collection chamber 16 is made. In one example, the length of the chamber is around 16 mm, and the chamber walls are formed from PMMA (Poly(methyl methacrylate)), and a suitable depth for the chamber is between 0.5 mm and 1 mm. However, for other constructions the chamber depth may be different.

When the blood 29 reaches the inner end 20 of the collection chamber 16, at the junction between the collection chamber 16 and the retention chamber 21, the flow of blood 29 stops. This will arise from surface tension acting at the step between the relatively small depth of the collection chamber 16 and the relatively large depth of the retention chamber 21.

This situation is shown in FIG. 5b. At this stage, the collection chamber 16 is full or substantially full of blood, but no blood has entered the retention chamber 21.

The skilled reader will appreciate that, as blood flows into the collection chamber 16, it will displace air which was previously in the collection chamber 16, and air will flow out of the cuvette 1 through the communication passages 24.

As a result of this, the user may simply touch and hold the apex 10 of the cuvette 1 to a quantity of blood 29, and the blood will flow in through the ingress aperture 18 to fill the collection chamber 16, and flow will then automatically stop. Once the user has done this, the user may lift the cuvette 1 away from the quantity of blood, and the collection chamber 16 will remain full of blood. The blood will not escape from the ingress aperture 18 because it will be held in place by capillary action, and it will also not (as discussed above) flow from the collection chamber 16 into the retention chamber 21.

The cuvette 1 is therefore configured to collect a fixed and known quantity of blood (i.e. equal to the volume of the collection chamber 16), in an easy and reliable manner.

Once blood has been gathered into the collection chamber 16 in this manner, the cuvette 1 may be placed into a centrifuge (not shown). The cuvette 1 will be arranged in the centrifuge so that the apex 10 of the cuvette 1 lies closest to the axis of rotation, with the analysis chamber 26 extending generally radially away from the axis of rotation.

When the cuvette is centrifuged, the blood 29 will be driven in a direction towards the rear end 4 of the cuvette 1. This force will be sufficient to overcome the forces of surface tension which, until this point, held the leading edge of the blood 29 at the inner end 20 of the collection chamber 16. The blood 29 will therefore flow into the retention chamber 21, and then from the retention chamber 21 into the junction region 23, and into the top end 28 of the analysis chamber 26. This situation is shown in FIG. 5c.

As centrifugation continues, the blood 29 is driven into the analysis chamber 26. As discussed above, the volume of the analysis chamber 26 is slightly greater than that of the collection chamber 16, and so the entire sample of blood 29 that has been collected will fit into the analysis chamber 26. This will leave the collection chamber 16, retention chamber 21, junction region 23 and a region at the top end 28 of the analysis chamber 26 free or substantially free from blood 29. This position is shown in FIG. 5d.

Once the blood 29 has fully transferred into the analysis chamber 26, it can be further centrifuged and analysed. For instance, one or more light sources may be positioned on one side of the cuvette 1, with one or more light detectors positioned either on the same side of the cuvette 1 (to receive light from the light sources by reflection) or on the other side of the cuvette 1 (to receive light from the light sources that is transmitted through the cuvette 1 and blood sample).

One advantage of the whole sample of blood collected into the collection region 16 fitting fully into the analysis chamber 26 is that all components of the blood 29 that has been collected are therefore present in the analysis chamber 26 to be analysed. For instance, a user may wish to analyse the blood sample to determine the volume of red blood cells therein, as a proportion of the volume of the entire sample. As a result of centrifugation, the red blood cells (as the densest component of the blood) will be forced to the bottom end of the analysis chamber 26. The red blood cells will absorb more light than other components of blood. The skilled reader will readily understand how the analysis of other components of blood, such as white blood cells and platelets, may be carried out.

All components of the blood will absorb light as opposed to regions where there is no blood. Therefore, analysing the length of the region in the analysis chamber 26 over which light intensity is low due to absorption by the blood will determine the overall volume of the blood sample (since the depth and width of the analysis chamber 26 are known), and the length of the analysis chamber 26 over which light is absorbed strongly will give the volume of the red blood cells.

As discussed above, when blood initially flows into the collection chamber 16, it will stop at the inner end 20 thereof, as a result of surface tension. FIG. 6 shows a close-up view of the inner end 20 of the collection chamber 16, where the collection chamber 16 meets the retention chamber 21. As can be seen in this figure, the collection chamber 16 communicates directly with the retention chamber 21, with no intervening chambers or other features.

FIG. 6 shows a cut-away side view. In this example, the front wall 33 of the retention chamber 33 (i.e. the wall closest to the collection chamber 16) is exactly or substantially perpendicular with the plane of the main body 2. Where the inner surfaces 17 of the collection chamber 16 meet the front wall 33 of the retention chamber, therefore, an angle 34 of around 90° is formed.

Where the blood 29 meets this angle 34, surface tension in the blood prevents the blood from passing into the retention chamber 21. Where the blood 29 stops at the inner end 20 of the collection chamber 16 it may form an indented shape 35, which protrudes inwardly into the collection chamber 16 by a short distance.

The front wall 33 of the retention chamber 21 need not be perpendicular to the plane of the main body 2. However, if the angle 34 is too large, then the surface tension effect discussed above will not occur, and the blood 29 will not stop at the inner end 20 of the collection chamber 16. It is envisaged that this effect may not occur for angles above around 135°.

The presence of a sharp corner between the inner surface 17 of the collection chamber 16 and the front wall 33 of the retention chamber is also preferred in order to give rise reliably to the desired surface tension effect. A rounded or chamfered corner at this point should preferably be avoided.

The retention chamber 21 is deeper than the collection chamber 21. As shown in FIG. 6, there is a distance 36 between the each inner surface 17 of the collection chamber 16, and the corresponding inner surface 37 of the retention chamber 21.

The retention chamber 21 also has a length 38, in a direction generally parallel with the plane of the main body 2, between the front wall 33 thereof and the far end of the retention chamber 21, i.e. the first feature (not shown in FIG. 6) which is aligned with the retention chamber 16.

As discussed above, FIG. 6 shows a side view, i.e. through the depth of the cuvette 1. As can be seen in FIG. 3, the retention chamber 21 is also wider, in a direction passing across the width of the main body 2, than the inner end 20 of the collection chamber 16.

As discussed above, it is envisaged that where the blood 29 stops at the inner end 20 of the collection chamber 16, it will form an indented shape 35. However, under certain circumstances it is possible that the blood will form a bulge, which protrudes into the retention chamber by a short distance. If so, it is important that the dimensions of the retention chamber 21, particularly the step height 36 and the length 38, are sufficient to prevent any part of the bulge from contacting any of the inner walls of the retention chamber 21.

Returning to FIGS. 1 and 2, in a preferred embodiment the main upper and lower surfaces 8, 9 of the main body 2 of the cuvette 1 are preferably formed to have a frosted or textured finish. This assists with easy gripping and handling of the cuvette 1 by a user, and also makes the otherwise transparent cuvette 1 easier to see.

However, in the example shown the cuvette 1 has, on each side, two regions where there is no frosting or texturing, and the cuvette 1 is formed to have a generally smooth, flat surface which transmits light effectively.

The first region 30 generally overlies the collection chamber 16, and may have a shape which is the same as, or similar to, the shape of the collection chamber 16. This first region 30 allows a user to see clearly that blood has been successfully drawn into the cuvette 1 when it is first used.

A second region 31 overlies the analysis chamber 26. This second region 31 allows effective analysis of blood 29 which is held in the analysis chamber 26, for instance during centrifugation.

In the embodiment shown, the collection chamber 16 has an “hourglass” shape, which, as it passes from the ingress aperture 18 at the front end 3 of the cuvette 1, tapers in width to a narrow point, and flares in width again to meet the retention chamber 21. In preferred embodiments, this shape functions as a Venturi constriction, which helps to accelerate the flow of blood as it is drawn into the front region of the collection chamber 16 from the ingress aperture 18.

The widening portion of the collection chamber 16, between the narrow point and its inner end 20, serves to slow the flow of blood again, so that as it reaches the junction between the collection chamber 16 and the retention chamber 21 it is moving at a relatively slow rate and will reliably be stopped at this junction by surface tension.

As discussed above, the various chambers may be created by indentations formed on the inner sides of one or both of the halves of the cuvette 1. In the example shown in the figures, indentations are formed in both halves to create the retention chamber 21, but indentations are only formed in one of the halves to create the collection chamber 16, junction portion 23 and analysis chamber 26. The skilled reader will appreciate that there are many other possibilities.

It is also envisaged that cuvettes embodying the present invention may have a series of internal chambers of any depth, i.e. not necessarily having a change in chamber depth that causes blood flow to stop through the effects of surface tension, as discussed above, but having one or more protrusions that make the cuvette easier to pick up from a flat surface. In such embodiments, the cuvette has a main body which is generally planar, and at least one protrusion which extends away from the main body in a direction which has a component that is perpendicular to the plane of the body. The result of the protrusion is that, when the main body is placed on a flat surface, at least a part of the main body is raised upwardly from the surface by at least 1 cm, i.e. there is at least 1 cm of space between the main body and the flat surface, into which a user can insert a finger to grasp and lift the cuvette easily. Any of the other features of the protrusion(s) described above may also be included.

The skilled reader will appreciate that cuvettes embodying the present invention may be easy to handle and operate by a home user, and will provide significant advantages with respect to known designs of cuvette.

When used in this specification and the claims, the term “comprises” and “comprising” and variations thereof mean that specified features, steps or integers and included. The terms are not to be interpreted to exclude the presence of other features, steps or compounds.

The features disclosed in the foregoing description, or the following claims, or the accompanying drawings, expressed in their specific forms or in terms of a means for performing the disclosed function, or a method or process for attaining the disclosed result, as appropriate, may, separately, or in any combination of such features, be utilized for realising the invention in diverse forms thereof.

1. A cuvette for collecting a blood sample, the cuvette comprising:
a main body;
a collection aperture formed within the main body;
an ingress aperture, allowing communication between a first end of the collection aperture and an exterior of the cuvette; and
a retention chamber formed within the main body and adjacent a second end of the collection chamber, which is generally opposite the first end thereof, wherein:
the collection chamber has a first depth, which is the same or substantially the same across the collection chamber, from the first end to the second end thereof; and
the retention chamber has a second, greater depth.

2. The cuvette according to claim 1 wherein, in a region where the collection chamber meets the retention chamber, the collection chamber is generally planar, and the retention chamber has a depth that is greater than that of the collection chamber on both sides of the plane of the collection chamber.

3. The cuvette according to claim 1 wherein, in a region where the collection chamber meets the retention chamber, the collection chamber is generally planar, and the retention chamber has a width, in a direction generally parallel with the plane of the collection chamber, which is greater than a width of the second end of the collection chamber.

4. The cuvette according to claim 1, wherein the collection chamber has a pair of sidewalls.

5. The cuvette according to claim 4, wherein a width of the collection chamber, defined between the sidewalls thereof, is greater at the first end of the collection chamber than at a middle portion thereof.

6. The cuvette according to claim 5, wherein the width of the collection chamber is greater at the second end thereof than at the middle portion.

7. The cuvette according to claim 1, further comprising one or more communication passages, which are in communication with the retention chamber at a first end thereof, and are in communication with the exterior of the cuvette at a second end thereof.

8. The cuvette according to claim 7, comprising two communication passages.

9. The cuvette according to claim 8, wherein the second ends of the two communication passages are on either side of the ingress aperture.

10. The cuvette according to claim 1, further comprising an analysis chamber, which is in communication with the retention chamber at a first end thereof.

11. The cuvette according to claim 10, wherein the analysis chamber is enclosed on all or substantially all sides, apart from the first end thereof.

12. The cuvette according to claim 10, wherein the analysis chamber is elongate, and is of constant or substantially constant width along its length.

13. (canceled)

14. The cuvette according to claim 10, wherein a volume of the analysis chamber is the same as, or greater than, a volume of the collection chamber.

15. The cuvette according to claim 10, wherein the cuvette further comprises one or more communication passages, which are in communication with the retention chamber at a first end thereof, and are in communication with the exterior of the cuvette at a second end thereof, further comprising a junction region adjacent the retention chamber, the analysis chamber and one or more communication passages being in communication with the junction region.

16. The cuvette according to claim 1, wherein dimensions of the ingress aperture and collection chamber are such that, when the ingress aperture is touched to a blood sample, blood is drawn into the collection chamber through capillary action.

17. The cuvette according to claim 16, wherein dimensions of the collection chamber and retention chamber are such that blood drawn into the collection chamber through capillary action will stop when it reaches the retention chamber, and will not be drawn into the retention chamber.

18. The cuvette according to claim 1, wherein the main body is generally planar, and the cuvette further includes one or more protrusions which extend away from a plane of the main body, such that when the cuvette is placed on a planar surface, at least one part of the main body is raised above the surface by a distance which is sufficient to allow a person to grasp the cuvette.

19. (canceled)

20. (canceled)

21. The cuvette according to claim 18, wherein the or each protrusion extends away from the plane of the main body in both directions.

22. A method of collecting a blood sample, comprising the steps of touching the ingress aperture of a cuvette according to claim 1 to a blood sample, such that blood is drawn into the collection chamber of the cuvette by capillary action, to fill or substantially fill the collection chamber, without substantially entering the retention chamber.

23. The method according to claim 22, wherein the cuvette further comprises an analysis chamber, which is in communication with the retention chamber at a first end thereof, and the method further comprises the step of centrifuging the cuvette so that the blood in the collection chamber flows through the retention chamber and into the analysis chamber.
